(19) United States
(12) Patent Application Publication
(10) Pub. No.: US 2019/0008465 A1
(43) Pub. Date: Jan. 10, 2019

(54) GENERATING AN INDICATOR OF A CONDITION OF A PATIENT

(71) Applicant: KONINKLIJKE PHILIPS N.V., Eindhoven (NL)

(72) Inventors: JENS MUEHLSTEFF, AACHEN (DE); RALPH WILHELM CHRISTIANUS GEMMA RSA WIJSHOFF, MUNSTERGELEEN (NL)

(21) Appl. No.: 15/750,567

(22) PCT Filed: Jul. 25, 2016
(86) PCT No.: PCT/EP2016/067603

§ 371 Date: Feb. 6, 2018

(30) Foreign Application Priority Data
Aug. 7, 2015 (EP) 15180194.1

(51) Int. Cl. A61B 5/00; A61B 5/117; A61B 5/0205; G16H 50/20

(52) U.S. Cl. CPC A61B 5/7282; A61B 5/117; A61B 5/74; A61B 5/01; G16H 50/20; A61B 2560/0242; A61B 5/02055

(57) ABSTRACT

A method (3000) of indicating a condition of a patient is disclosed. The method comprises: receiving (3100), on a processor, vital signs information from a sensor for monitoring vital signs of the patient; receiving (3200), on said processor, environmental and/or contextual information for the patient from an environmental sensor for monitoring environmental and/or contextual information; communicating (3300) the environmental and/or contextual information to a database that stores a plurality of patient-specific reference values, each reference value corresponding to a different environmental and/or contextual condition; selecting (3400), on said database, a patient-specific reference value among the said plurality in response to the received environmental and/or contextual information; receiving (3500), on said processor, the selected patient-specific reference value for the vital signs information of the patient from said data-base; and processing (3600), on said processor, the vital signs information and the selected patient-specific reference value so as to generate an indicator of a condition of the patient.

### FIELD OF THE INVENTION

This invention relates to the field of indicating a condition of a patient, and in particular to the field of processors adapted to generate an indicator of a patient's condition.

### BACKGROUND OF THE INVENTION

In many environments, in particular in emergency care or first aid, it is desirable for an indication of a patient's condition or state of health to be quickly and easily generated based on a measured vital sign.

In standard medical practice, there are at least four acknowledged vital signs of an individual including: blood pressure, heart rate, body temperature, and respiratory rate. That being said, other vital signs of a patient are well known, for example, peripheral arterial oxygen saturation (SpO2) and blood glucose level.

It is known to measure a current value of a vital sign of a patient and compare said current value to a threshold or critical value of the vital sign. This may, for example, be used to generate an indicator of a condition of a patient. However, reliable measurements of vital signs may not be easy or possible to obtain, especially in emergency situations where medical equipment and/or medically-trained persons are not available.

Document US 2014/257058 A1 describes an automated personal medical diagnostic system that includes a sensor configured to measure and/or sense a physiological condition and generate or acquire sensor data, a computing device configured to process the sensor data and generate diagnostic data (e.g., medical diagnostic data, trigger data, or any combination thereof) based on the sensor data, and a user interface configured for user interaction.

### SUMMARY OF THE INVENTION

The invention is defined by the independent claims. Advantageous embodiments are provided by the dependent claims.

According to a first aspect of the inventive concept there is provided a method of indicating a condition of a patient, the method comprising: receiving, on a processor of a portable computing device, vital signs information from at least one sensor for monitoring vital signs of the patient; receiving, on said processor, environmental and/or contextual information for the patient from at least one environmental sensor for monitoring environmental and/or contextual information; communicating the environmental and/or contextual information for the patient from said processor to a database that stores a plurality of patient-specific reference values, each reference value corresponding to a different environmental and/or contextual condition; selecting, on said database, a patient-specific reference value among the plurality of patient-specific reference values in response to the environmental and/or contextual information for the patient received from said processor; receiving, on said processor, the selected patient-specific reference value for the vital signs information of the patient from said database; and processing, on said processor, the vital signs information and the selected patient-specific reference value so as to generate an indicator of a condition of the patient.

In other words, there is herein described a method of generating an indicator of a patient's condition by processing vital sign information and a selected patient-specific reference value (or selected patient-specific reference information) for the vital sign information. An indicator of a patient's condition may therefore be obtained by processing a value of a vital sign based on the selected patient-specific reference value for the vital sign information (e.g. an expected, anticipated or historically normal value, or characteristics of the patient such as height, weight, age, fitness, etc.). That is to say, the method may comprise receiving a current value of a vital sign (e.g. from a sensor) and a selected patient-specific reference value (e.g. from a database) for the vital sign. An indicator of a condition of the patient may then be generated based on the vital sign value(s) and the selected patient-specific reference value, thereby accounting for patient-specific characteristics of the vital sign that may otherwise be misleading when compared or considering against generic or standard threshold values that do not account for the patient's unique circumstances or characteristics. Also, a link between a vital sign and patient-specific reference information (such as height and weight for example) may be known and used to derive an indicator or patient condition from a vital sign measurement that may have otherwise been useless on its own to an unskilled person.

Use of a patient-specific reference value in the generation of an indicator may reduce inaccuracy in indicating a patient's condition, since the processing may account for known characteristics or ‘normal’ medical conditions of a patient (e.g. low resting heart rate, or high blood pressure). Hence, generating an indicator of a condition of a patient based on both a sensed vital sign information (e.g. a measured value) and patient-specific reference values or information (e.g. height, weight, age, fitness, etc.) may allow for a more accurate and reliable indicator of the patient's condition.

The patient-specific reference value may therefore be used to effectively calibrate or adjust a measured current value of a vital sign for subsequent comparison with a condition characterizing value (e.g. threshold or alarm value). In other words, the method may comprise calibrating or otherwise adjusting a sensed value of a vital sign using the selected patient-specific reference value so as to generate an adjusted (or ‘calibrated’) value specific to the patient. This patient-specific value may be subsequently compared with a generic or industry-standard threshold value, and so the patient-specific value may be thought of as being an indicator of the patient's condition which is adapted to take account of patient-specific characteristics (such as historical and/or current health traits, for example).

It will therefore be understood that generation of the indicator of a condition of a patient may also be performed with reference to a condition characterizing value. In other words, the indicator may be generated further based on a condition characterizing value which is indicative of a condition of the patient (e.g. a threshold value of a vital sign above which a patient's condition is deemed dangerous). The condition characterizing value may be a standardised or average value of a vital sign, such that it is not dependent upon the patient's unique circumstances or characteristics. By providing a standardised measure of the vital sign (independent of the current or patent-specific reference values) a high accuracy of the indicator of a patient's condition may be maintained. Furthermore, a single condition characterizing value may be readily and easily updated to reflect new trends in condition identifying values without, for example, necessitating the need to determine a plurality of condition characterizing values for different patients.

The indicator of a condition of the patient may, for example, be helpful for enabling a lay-person (e.g. a non-medically trained individual) to take appropriate action in the event of an emergency situation. Medical guidance may therefore be generated based on an indicator of a patient's condition obtained using a proposed embodiment. Embodiments may therefore further comprise generating medical guidance (e.g. instructions) to a person based on the generated indicator of a condition of the patient.

In some embodiments, processing the vital signs information and the selected patient-specific reference value further comprises processing the environmental and/or contextual information for the patient. In other words, the method may further consider environmental or contextual information in the generation of an indicator of a condition of a patient.

The environment of a patient may, for example, affect a current measured value of a vital sign such that the current measured value may not provide a true or direct representation of the patient's condition. For example, a change in altitude may influence SpO2 readings. Accordingly, the method may advantageously account for such changes in environmental or contextual differences when generating an indicator.

The environmental or contextual information may comprise, for example, at least one of the following: location information; weather information; and hazard information.

Location information may include, for example, information about the immediate location such as temperature, altitude, humidity or vibrations. Weather information may comprise information about past, present or predicted weather conditions, for example, whether it has recently rained. Hazard information may comprise information relating to gases (e.g. high CO or low O₂ levels), proximate chemicals and/or noises (e.g. high noise levels or noises made by the patient).

According to the first aspect of the invention, the method comprises communicating the environmental and/or contextual information for the patient to the database, so as to enable the patient-specific reference value for vital signs information received to be environmentally and/or contextually specific. Advantageously, the database stores a plurality of different reference values for a specific vital sign of the patient, each reference value relating to a different environmental and/or contextual condition (e.g. a measurement of a patient's heart rate at different temperatures). By passing the environmental condition to the database, the processor allows the possibility of environmentally and/or contextually-specific reference values for a vital sign to be returned, so as to improve the accuracy and reliability of the indicator of a patient's condition.

In some embodiments, selecting the patient-specific reference value comprises determining an environmental and/or contextual condition based on the received environmental and/or contextual information, and selecting a stored patient-specific reference value corresponding to an environmental and/or contextual condition that is closest to the determined environmental and/or contextual condition. In order to determine the proximity or closeness of a determined environmental and/or contextual condition to one of the environmental and/or contextual conditions stored in the database, the method may advantageously use a correlation or a classification algorithm, such as k-means clustering.

In some embodiments, the database is provided by a remote portable computing device carried by the patient. In other or further embodiments, the database may be provided on a memory of the portable computing device or in a central database accessible by interconnected networks (that is a database stored on an external server accessible, for example, via the internet).

In other words, the database may be stored either on the portable computing device or external to the portable computing device. A remote portable computing device carried by the patient (e.g. a mobile phone) may store a database comprising information related to the reference value of the vital sign. In other embodiments, the database is stored on an external server, for example a server in a data centre. Such servers may only be accessible via the internet, for example, such that the database may be considered to be stored in ‘the cloud’.

In some embodiments, a combination of databases may be used, for example, a patient-specific database may be stored on a remote portable computing device, with a ‘back-up’ database storing, for example, generic values for a patient being stored on the portable computing device. In other words, if a patient, for example, does not carry a remote portable computing device storing patient-specific reference information which may be received by the processor, the processor may instead receive patient-specific reference information from a database stored on the processor. The database stored on the processor may comprise generic reference values for a patient, but may also be based on identifying characteristics of the patient (e.g. based on age, height, gender etc.)

The method optionally further comprises receiving, on said processor, identification information of the patient for identifying the patient. The method may further comprise passing the identification information to the database, so as to enable the selected reference value for the vital signs information received to be patient-specific. For example, the portable computing device may be adapted to receive a characteristic of the patient (for example, an age, a gender, a unique identity from a device carried by the patient and/or biometric data).

By providing the database with identification information, a database may be able to cross-reference such information with patient-specific reference information. For example, a unique code or identity carried by a device of the patient may correlate to patient-specific reference information (e.g. reference values unique to that patient) on the database. By receiving the identification information on the processor, the processor may advantageously provide the identification information to the database so as to enable patient-specific reference values to be provided, such patient-specific reference information thereby improving the accuracy and reliability of the indicator of the patient's condition (as specific known conditions of the patients are more accurately accounted for).

Embodiments may comprise providing the database with both environmental/contextual information and identification information of the patient. The database may store a plurality of different environmental-specific reference values for a specific patient (i.e. a patient identified using the identifying information may have a plurality of stored reference values corresponding to different environmental conditions).

According to a second aspect of the inventive concept, there is provided a computer program product comprising computer program code means adapted to perform all of the steps of any previously described method when said program is run on a portable computing device having a processor.

According to a third aspect of the inventive concept, there is provided a medical guidance system for indicating a condition of a patient, the system comprising a portable computing device and a database accessible to the portable computing device, wherein the database stores a plurality of patient-specific reference values for vital signs information of the patient, each reference value corresponding to a different environmental and/or contextual condition. The portable computing device comprises a processor adapted to: receive vital signs information from at least one sensor for monitoring vital signs of the patient; receive environmental and/or contextual information for the patient from at least one environmental sensor for monitoring environmental and/or contextual information; transmit the environmental and/or contextual information for the patient to the database; receive selected patient-specific reference value for the vital signs information of the patient from the database; and process the vital signs information and the selected patient-specific reference value so as to generate an indicator of a condition of the patient. The database is adapted to: receive the environmental and/or contextual information for the patient from the processor; select a patient-specific reference value among the plurality of patient-specific reference values in response to the received environmental and/or contextual information for the patient; and transmit the selected patient-specific reference value for the vital signs information of the patient to the processor. By factoring patient-specific reference information (such as the medical history of a patient) when determining an indication of a patient's condition, the quality and/or accuracy of the indicator is potentially improved.

The processor may be further adapted to process the vital signs information, the selected patient-specific reference value and the environmental and/or contextual information so as to generate an indicator of a condition of the patient.

Optionally, the database may be further adapted to determine an environmental and/or contextual condition based on the received environmental and/or contextual information, and select a stored patient-specific reference value corresponding to an environmental and/or contextual condition that is closest to the determined environmental and/or contextual condition.

The portable computing device may further comprise a patient identification unit adapted to obtain identification information for identifying the patient. The processor may optionally be adapted to pass the identification information to the database, such that the selected reference value for vital signs information received is patient-specific.

In at least one embodiment, the portable computing device further comprises at least one sensor adapted to monitor a vital sign of the patient. The sensor may, for example, comprise at least one of the following: an oximeter, a heart monitor, an EKG monitor, an electrode for positioning on a patient's skin; and/or a respiratory monitor. Such vital signs may be particularly relevant to the physical condition of a patient. This may facilitate the capturing of a wider variety of vital signs information for a patient, such that a patient's condition may be established at a higher level of confidence, thus further improving indicator quality and/or accuracy.

The portable computing device may additionally comprise an automatic defibrillator adapted to automatically defibrillate a patient based on the indicator of the condition of the patient. The automatic defibrillator may comprise one or more pads for positioning on a patient so as to deliver a shock to the patient and/or act as a heart rate sensor. This may obviate the need for an initial human intervention-based emergency treatment process, in particular if the patients are already known to the system. This therefore has the potential to further reduce the amount of human intervention required in an emergency (e.g. sudden cardiac arrest) situation or patient triage system.

These and other aspects of the invention will be apparent from and elucidated with reference to the embodiment(s) described hereinafter.

### BRIEF DESCRIPTION OF THE DRAWINGS

Embodiments of the invention are described in more detail and by way of non-limiting examples with reference to the accompanying drawings, wherein:

FIG. 1 is a schematic block diagram of a medical guidance system according to an embodiment of the invention;

FIG. 2 is a schematic diagram of a medical guidance system according to another embodiment of the invention;

FIG. 3 illustrates the function of a medical guidance system according to an embodiment in an emergency care scenario;

FIG. 4 illustrates the function of a medical guidance system according to another embodiment in an emergency care scenario;

FIG. 5 is a graph which showing plots of normalised pulse arrival time (on the Y-axis) versus normalised Heart Rate (on the X-axis) for a plurality of patients; and

FIG. 6 is a flow diagram of a method of an example embodiment of a method of generating an indicator of a condition of a patient.

### DETAILED DESCRIPTION OF THE EMBODIMENTS

It should be understood that the Figures are merely schematic and are not drawn to scale. It should also be understood that the same reference numerals are used throughout the Figures to indicate the same or similar parts.

The invention provides a concept for generating an indicator of a condition of the patient by using a portable computing device, and a database accessible to the portable computing device, to process vital signs information of the patient in combination with patient-specific reference values for the vital signs information. By employing patient-specific reference values, measured values of a patient's vital sign(s) may be adjusted or tailored based on characteristics of the patient so as to provide an indicator which accounts for one or more factors specific to the patient (such as the patient's physical characteristics and/or medical history for example). In this way, a higher quality (e.g. more accurate) indicator of condition may be obtained which accounts for characteristics that are unique or specific to the patient.

The portable computing device of the medical guidance system may comprise a smartphone, smartwatch, personal digital assistant or tablet computer. Also, the portable computing device may be a head-mountable computing device having at least one display module arranged to be viewed by the wearer of the head-mountable computing device when wearing the device. Embodiments may thus employ conventional and widely-available portable computing devices that are commonly carried or worn by a significant number of people. Moreover, as there is a clear trend in society towards smart portable computing devices, it is reasonable to assume that a significant number of lay persons will be in the possession of such a portable computing device, thereby increasing the likelihood of a person being able to implement a method of generating an indicator of a patient's condition according to an embodiment. Furthermore, the portable computing device may be portable medical equipment, such as a (transport) patient monitor, a monitor/defibrillator combination, or an automatic external defibrillator (AED).

Embodiments may therefore utilize the insight that lay person rescuers or bystanders may carry (smart) portable computing devices that are capable of generating an indicator of a patient's condition from vital signs information and patient-specific reference values for the vital signs information. In this way, a medical guidance system may be compiled in situ by integrating one or more portable computing devices with at least one sensor for monitoring vital signs of the patient in an automated or user-controller manner. In this manner, devices having the desired sensing and/or processing capability can be configured such that they can be included into a medical guidance system according to an embodiment. Based on generating an indicator of a patient's condition, the system may communicate medical guidance to a user. This may enable a user to provide suitable and/or appropriate medical attention, despite the user have no medical training and/or no specialised medical equipment, thus helping to ensure that the patient receives appropriate care or treatment.

The term patient should not necessarily be constructed as limited to an individual in a critical condition or in a clinical environment, but may rather include any individual for whom it may be desired to measure a vital parameter (e.g. an athlete and/or jogger). That being said, in preferable embodiments, the sensor system and/or portable computing device may be designed for use in an emergency care situation.

In the context of the present application, a portable computing device is a computing device that may be worn or carried by a person. Typically, such a portable computing device may be equipment suitable for connection to a vital sign sensor (e.g. a pulse oximeter). Examples of portable computing devices include mobile phones, smart-watches, tablet computers, personal digital assistants, and laptop personal computers.

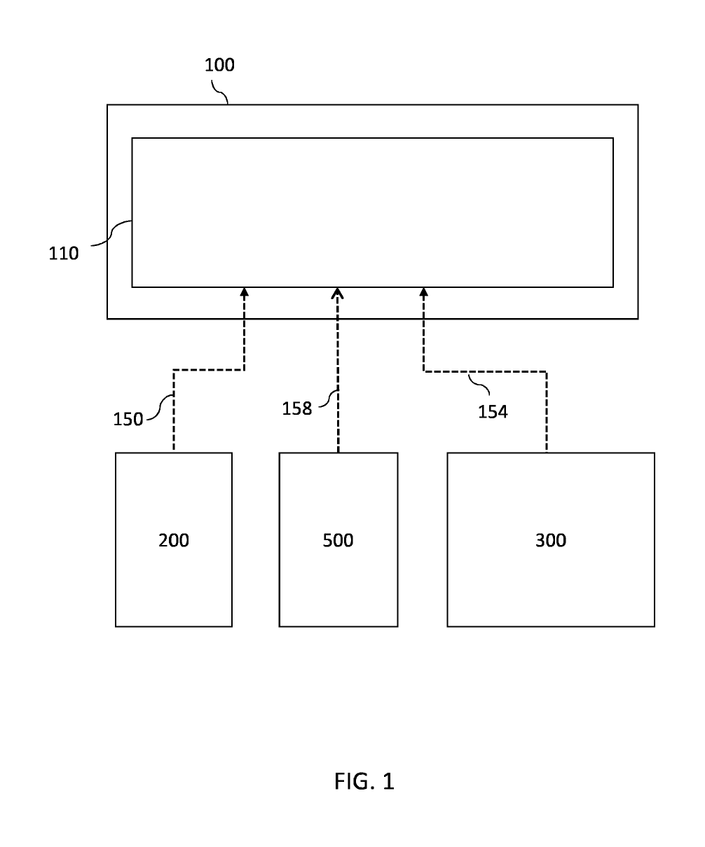

The basic structure and operation of a portable computing device 100 according to a first embodiment of the invention may be described with reference to FIG. 1. FIG. 1 is a schematic block diagram of a system for generating an indicator of a condition of a patient, which may be useful for providing medical guidance for example.

The portable computing device 100 comprises a processor 110. The processor is adapted to receive a current value 150 of a vital sign from at least one sensor 200 adapted to monitor the vital sign of the individual. The processor 100 is adapted to receive environmental and/or contextual information 158 from an environmental sensor 500. The processor is further adapted to receive reference values 154 for that vital sign from a database 300.

Sensors suitable for generating a current value for a vital sign are well known in the prior art and may comprise, for example, an oximeter, a heart rate monitor, a blood pressure monitor, an EKG monitor, a respiratory monitor and/or an EKG monitor.

The processor 110 is adapted to process the received current value (from the at least one sensor) and a reference value (from the database) so as to generate an indicator of a condition of a patient. The processor may also generate the indicator of a patient's condition further based on environmental and/or contextual information.

The indicator may, for example, be a representative value of the condition of a patient (e.g. severity on a scale of 1-10) or a binary indicator.

In processing the received current value and the reference value, the processor 110 may, for example, calibrate a current measurement of a vital sign based on a received reference value of that vital sign. The processor may then compare this calibrated value to a standardised condition characterizing value (i.e. a critical or threshold value) of that vital sign to determine an indicator of a condition of a patient. Calibration of the current measurement based on the received reference value thereby allows for an accurate and reliable method of determining a condition of the patient without necessitating the need for storing a plurality of condition characterizing values (e.g. threshold values) for the vital sign.

By way of example, the processor 110 may receive a current measurement 150 of a patient's present heart rate and a reference measurement 154 of a patient's resting heart rate. The processor may then calibrate the current measurement based on the reference measurement so as to ‘normalise’ the current measurement. For example, if a patient is known to have a low resting heart rate (e.g. <55 BPM) compared to the general population, a current measurement of the patient's heart rate may be calibrated upwards. The processor then generates an indicator of a patient's condition based on the calibrated measurement and an operation (e.g. a comparison) with a standardised condition characterizing value.

In other embodiments, the processor may determine a ratio of a current measurement of a patient's vital sign to the reference measurement of the patient's vital sign (e.g. current heart rate divided by reference heart rate). The processor may compare the determined ratio to a critical value of the vital sign to generate an indicator of the condition of a patient (e.g. if the ratio is lower than a critical value, a positive indicator may be generated).

In at least one embodiment, the current value of the vital sign may have no contextual significance without calibration or comparison to the patient-specific reference value for a vital sign. For example, the current value may be an indirect or surrogate parameter of a vital sign (for example, pulse arrival time, PAT) which, on its own, has no contextual significance. In other words, such surrogate measures only provide a reliable detection if compared to a healthy status of an individual. Thus, it has been advantageously herein recognised that a processor adapted to receive both a current value of a vital sign and a patient-specific reference value for that vital sign can allow an indicator of the vital sign to be reliably produced.

Such surrogate or indirect parameters for a vital sign (capable of being non-invasively measured) may comprise at least one of the following: pulse arrival time; pulse wave velocity; and pulse transit time.

The patient-specific reference value of the vital sign may preferably be based on identification information of the patient and may therefore be unique to that patient. In some embodiments, the patient-specific reference value may be obtained by comparison of characteristics of the patient with average reference values (e.g. the reference value may be based on a patient's gender and/or age).

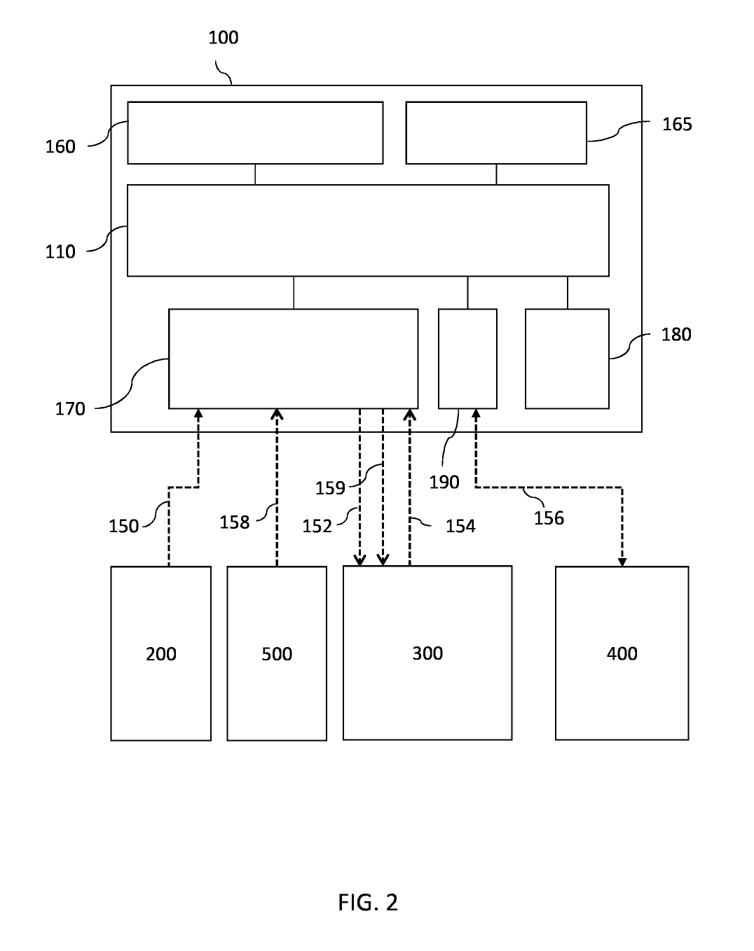

The current value of a vital sign received by the processor is preferably obtained using a non-invasive measure, and may, for example, comprise a measure of a patient's: heart rate; pulse arrival time; pulse wave velocity; pulse transit time; and/or peripheral arterial oxygen saturation (SpO2). A more complex medical guidance system according to a second embodiment of the invention may be described with reference to FIG. 2. Only those features of the system which are not yet described or differently embodied shall be hereafter described.

FIG. 2 is a schematic block diagram of a system according to another embodiment of the invention. The system may be referred to as a medical guidance system since it may generate an indicator of a condition of a patient, and medical guidance may be generated by the system based on the indicator of condition.

FIG. 2 illustrates a portable computing device comprising a processor 110, a visual output device 160, audio output device 165, a communications unit 170, an identification unit 190 and a memory unit 180.

The portable computing device 100 is adapted to communicate with a number of devices, including at least one sensor 200, a database 300, a remote device 400 and an environmental sensor 500. The communication unit 170 may be adapted to receiver and/or transmit signals from/to at least one of these devices, and may thereby act as a transceiver. The communication unit 170 is adapted to interact with the processor 110 so as to convey signals to and from the processor 110 (i.e. the processor may receive information from the devices via the communication unit).

The portable computing device 100 is adapted to display a visual representation of the indicator of a patient's condition generated by the processor 110 on the visual output device 160. The visual output device 160 may comprise one or more LEDs adapted to identify a patient's condition (e.g. an array of LEDs forming a display or a series of one or more LEDs).

In other or further embodiments, the portable computing device comprises an audio output display 165 (e.g. a speaker or buzzer) adapted to provide an audio representation (e.g. a confirmation sound/voice or a buzz) indicating a patient's condition.

In one embodiment, the processor 110 further calibrates the current value of the vital sign based on the environmental information 158 received from the environmental sensor 500. In other words, the processor 110 may calibrate or amend the current value of the vital sign based on the received reference value of the vital sign and environmental information. This may, for example, allow a current value of a measured sign to be calibrated dependent upon environmental conditions.

For example, it might be known that an individual's resting heart rate may be dependent upon an external temperature, and accordingly the environmental sensor 500 may be a thermometer and the vital sign may be a heart rate. Accordingly, the processor may calibrate the current value of the heart rate downwards slightly in warm temperatures (e.g. >28° C.) and calibrate the current value of the heart rate upwards slightly in cooler temperatures (e.g. <18° C.). Thus, the current value for the heart rate may be substantially normalised to a consistent temperature (e.g. normalised to room temperature ˜23° C.).

In other or further embodiments, to perform the generation of an indicator of a patient's condition based on the environmental and/or contextual information, the processor may be adapted to pass (e.g. via the communication unit 170) the environmental and/or contextual information to the database as output environmental and/or contextual information 152.

The database 300 stores a plurality of reference values of a vital sign, each corresponding to a different environmental and/or contextual condition. Upon receiving the output environmental or contextual information 152 from the portable computing device 100, the database selects a reference value for an environmental and/or contextual condition closest to the environment of the portable computing device for transmittal to the portable computing device.

Thus, the portable computing device is adapted to send environmental and/or contextual information 152 to the database 300 so as to receive a reference value which is at least partially dependent upon the output environmental and/or contextual information. Environmental and/or contextual information may comprise, for example, location information, hazard information and/or weather information. Location information should be understood to mean information specific to the general vicinity of the portable computing device, for example: longitude and latitude; temperature; altitude; humidity; and/or air pressure. Weather information may comprise weather information specific to a broad vicinity of the portable computing device, for example, whether it has recently rained in the vicinity.

Hazard information may relate, for example, to hazards in the general vicinity of the portable device, for example, high levels of CO or low levels of O₂. The hazard information may comprise information concerning the proximity of certain chemicals or known hazards in the area (e.g. radiation levels). Accordingly, the environmental sensor may comprise any number of the following: a global positioning system (GPS); a thermometer; a hydrometer; an altimeter; barometer; a Geiger counter; a light sensitive sensor; and/or a gas sensor.

The identification unit 190 of the portable computing device is adapted to generate or receive identification information of the patient for identifying the patient.

The identification unit 190 may be adapted to communicate with a remote device 400 carried by the patient in order to receive identification information 156 of the patient from the remote device 400. The remote device may, for example, be a mobile phone, smartwatch, a chip integrated in a card (e.g. passport, driver's licence and/or medical insurance card), or a chip implanted in the patient's body storing a unique identifier which may be read by the identification unit. In some embodiments, the remote device may transmit a signal (e.g. a characteristic signal) which is received by the identification unit.

In alternative embodiments, the identification unit may be a scanner or biometric sensor adapted to generate identifying information of the patient based on biometric data (e.g. a fingerprint or iris scan) or a unique identifier on an object held by the patient (e.g. a barcode or a QR code).

The processor may be adapted to pass the identification information 159 to the database 300 so as to receive a patient specific reference value from the database. In other words, the database may store a plurality of different reference values of a vital sign, each reference value corresponding to a different patient. Identification information of the patient, transmitted by the portable computing device, may be received by the database such that the database may select a reference value unique to the patient. In this way, patient-specific reference values may be passed to the portable computing device.

This may advantageously improve the reliability and accuracy of the indicator of a patient's condition, as the reference value(s) (e.g. for calibrating the current value of the vital sign) may be chosen so as to be unique to an individual patient.

In some embodiments, the processor may be adapted to pass both environmental/contextual information 152 and the identification information 159 to the database 300 so as to receive a patient-specific and environmental and/or contextually-specific reference value from the database. In other words, the database may store a plurality of sets of reference values, each set corresponding to a different patient, and each reference value in that set corresponding to a different environmental and/or contextual condition. Accordingly, the accuracy of the reference value for the patient and the environment may be improved, so as to increase the accuracy and reliability of the indicator generated by the processor 110 of the portable computing device 100.

Optionally, the identification unit 190 may be formed as part of the communication unit 170, such that the communication unit may be adapted to communicate with a remote device carried by the patient in order to receive identification information of the patient from the remote device.

In some embodiments, the database may be internalised in the portable computing device 100 and may be stored, for example, on the memory unit 180 of the device.

In other or further embodiments, when the portable computing device is unable to receive a reference value from a database, a pseudo-reference value (e.g. an average or estimated reference value based on patient-specific characteristics) may be received from a back-up database stored on the memory unit 180 of the device, such that an indicator of a condition of the patient may be generated on the basis of the pseudo-reference value and the current value of the vital sign. Generation and/or calculation of the pseudo-reference value may be advantageously updated regularly to reflect changing trends in the characteristics of a population or medical advice so as to improve the accuracy of the indicator of the patient's condition.

The communication unit 170 and/or the identification unit may, for example, communicate with the database, sensor, environmental sensor or remote portable computing device by a direct wired connection using known wired protocols (e.g. a USB system) or wireless connection using known wireless protocols. Suitable wireless communication protocols that may be used to communicate with the remote portable computing device include an infrared link, Zigbee, Bluetooth, near-field communication (NFC), a wireless local area network protocol such as in accordance with the IEEE 802.11 standards, a 2G, 3G or 4G telecommunication protocol, and so on. Other formats will be readily apparent to the person skilled in the art.

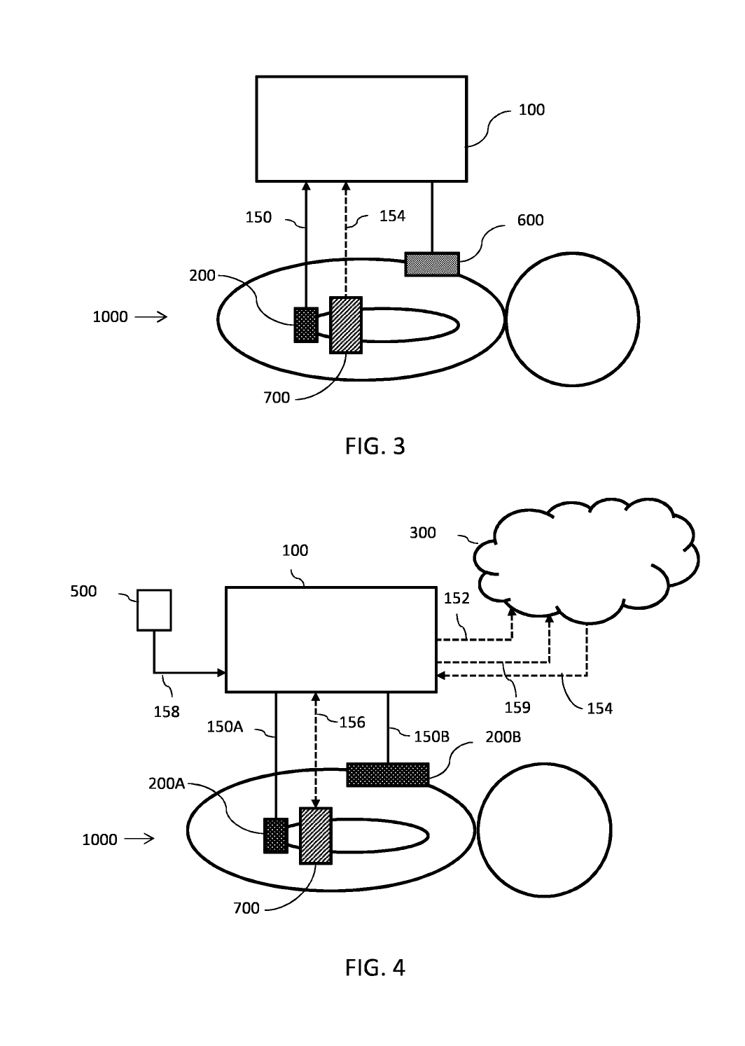

A third embodiment of a medical guidance system according to the invention may be described with reference to FIG. 3. Only those features of the medical guidance system which are not yet described or differently embodied shall be hereafter described. FIG. 3 illustrates the function of a medical guidance system according to the third embodiment in an emergency care scenario.

The portable computing device 100 is adapted to further comprise a sensor 200 and an AED 600 (automatic external defibrillator) both connected to a patient 1000. The patient carries a remote portable computing device, here embodied as a smartwatch worn on the patient's wrist.

The sensor 200 of the portable computing device is embodied as a heart rate monitor or oximeter adapted to detect a current value of a patient's heart or pulse rate (i.e. the vital sign measured by the sensor 200 is the patient's heart rate).

The remote portable computing device 700 is adapted to transmit a reference value of the heart rate of the patient to the portable computing device 100 (e.g. upon request by the portable computing device). For example, the remote portable computing device 700 may transmit information relating to the normal resting heart rate of the patient.

In other words, the remote portable computing device 700 carried by the patient holds or stores the database 300 from which the reference value of the vital sign is received.

The processor of the portable computing device is adapted to generate an indicator of the patient's condition based on, the received current value 150 of the heart rate (from the sensor 200) and the received reference value 154 of the heart rate (from the remote portable computing device 700).

The processor may be further adapted to determine, based upon the generated indicator, whether it is necessary for a patient to receive defibrillation. For example, the processor may be adapted to diagnose a state of the patient based on the indicator of the patient's condition, and determine whether to defibrillate based on the diagnosed state of the patient. The AED 600 is adapted to perform defibrillation based upon this determination.

Thus the portable computing device may be understood to be an automatic defibrillating system which, due to the received reference value 154, has an increased accuracy and reliability in determining an indicator of a patient, to thereby improve the reliability of the automatic defibrillating system.

A fourth embodiment of a medical guidance system according to the invention may be described with reference to FIG. 4.

The portable computing device 100 comprises an environmental sensor 500, first sensor 200A and second sensor 200B. First sensor 200A is adapted to provide a first current value of a vital sign 150A to the portable computing device, and second sensor 200B is adapted to provide a second current value 150B of the vital sign to the portable computing device 100.

In other words, the processor may receive a plurality of current values of vital signs from a plurality of sensors. In some embodiments, the plurality of current values may be combined or averaged so as to form a single current value which is used in the generation of the indicator of the patient's condition (e.g. take an average reading from more than one sensor).

The first sensor 200A may be an oximeter positioned on a patient's 1000 digit (e.g. finger) so as to detect a first reading 150A of the patient's pulse rate, and the second sensor 200B may be an electrode positioned on the patient's chest so as to take a second reading 150B of the patient's heart rate.

The portable computing device 100 is adapted to obtain identification information 156 of the patient 1000 using, for example, an identification unit of the portable computing device. The identification unit may be adapted to communicate with a remote portable device 700 carried by the patient (e.g. a smartwatch or bracelet containing an identifying RFID chip) so as to receive identifying information 156 of the user. In some embodiment, the portable device comprises an identifying design (e.g. a barcode or QR code) which the identification unit can identify.

The environmental sensor of the portable computing device is adapted to generate environmental or contextual information 158 that may be received by the processor of the portable computing device.

The portable computing device 100 is adapted to communicate with a database stored on an external server. The external server may be, for example, a server in a data centre and may be considered to be a ‘cloud storage system’. In other words, the portable computing device may be adapted to communicate with a database which is stored in a memory unit of a device not positioned in the vicinity of the portable computing device. Communicating with such a database may be performed via the internet or known wireless communication protocols.

The portable computing device 100 is adapted to pass the environmental/contextual information 152 and/or the identifying information 159 to the database so as to receive an environmental and/or contextual-specific and/or patient-specific reference value for generating an indicator of a patient's condition.

The portable computing device is then adapted to generate an indicator of the patient's condition based on, the readings of the pulse rate and heart rate (from the sensors 200A and 200B) and the received reference value 154.

During emergency situations, a reliable and fast non-invasive continuous blood pressure measurement is not available. However, blood pressure surrogate measures such as pulse transit time (PTT), pulse wave velocity (PWV) and pulse arrival time (PAT) are well-known. These are surrogate measures which do not provide absolute blood pressure, but do enable the determination of trends in blood pressure. The surrogate parameters can be easily measured non-invasively by easy-to-apply sensing modalities.

For PAT it has been shown that a similar discrimination between a patient's states of “save zone” and “critical zone” is possible by plotting PAT vs. Heart Rate in a 2D plot. However, surrogate measures may only provide a reliable detection if a measurement is related to the status of the subject.

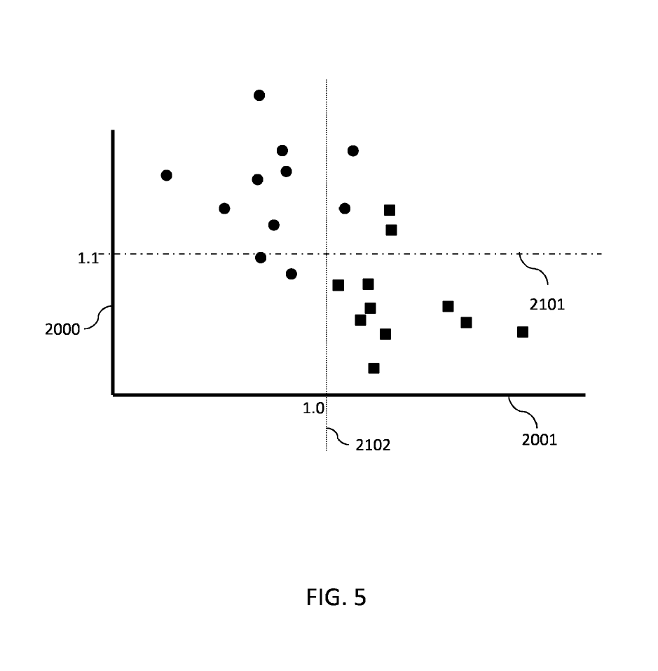

For example, this approach is demonstrated in FIG. 5 which shows a plot of normalised PAT (i.e. PAT/PATref) on the Y-axis 2000 versus normalised Heart Rate (i.e. HR/HRref) on the X-axis 2001.

Here, patient-specific reference values for PAT (i.e. PATref) and patient-specific reference values HR (i.e. HRref) are those which have been determined when a patient was/is in a stable, i.e., “healthy” state.

By using these reference values obtained in a “healthy condition”, critical blood pressure drops can be detected. This is illustrated in the graph of FIG. 5, where a comparison is shown between patients with a systolic blood pressure (SBP) below 80 mmHg (round dots, patients fainted) and patients with a SBP above this threshold (square dots, non-fainters). Thus, the square dots indicate conscious patients and the round dots indicate unconscious patients. These two groups can be discriminated clearly, when detecting a calibrated PAT higher than a threshold value of about 1.1 (as indicated by the horizontal line labelled 2101), accompanied by a low normalized heart rate, i.e. a normalised heart rate below 1.0 (as indicated by the horizontal line labelled 2102).

It will therefore be understood that condition characterizing values (e.g.

thresholds) using a plurality of vital sign measurements (e.g. both PAT and HR) that have been adjusted to account for patient-specific characteristics (e.g. normalised according to a patient-specific reference value) may provide an indication of a patient's condition.

At this point it is noted that a medical guidance system may be created in situ, using ubiquitous smart devices that are in the possession, e.g. worn or carried by a patient and/or bystanders. Given the rapid proliferation of such smart devices in everyday life, this therefore means that there is an excellent chance that such a medical guidance system according to an embodiment may be created in situ by identifying suitable components thereof at the scene of an emergency and incorporating the identified components in the medical guidance system, e.g. by establishing wireless links between the identified devices and the portable computing device, and by placement of the identified devices on and/or near the patient if necessary.

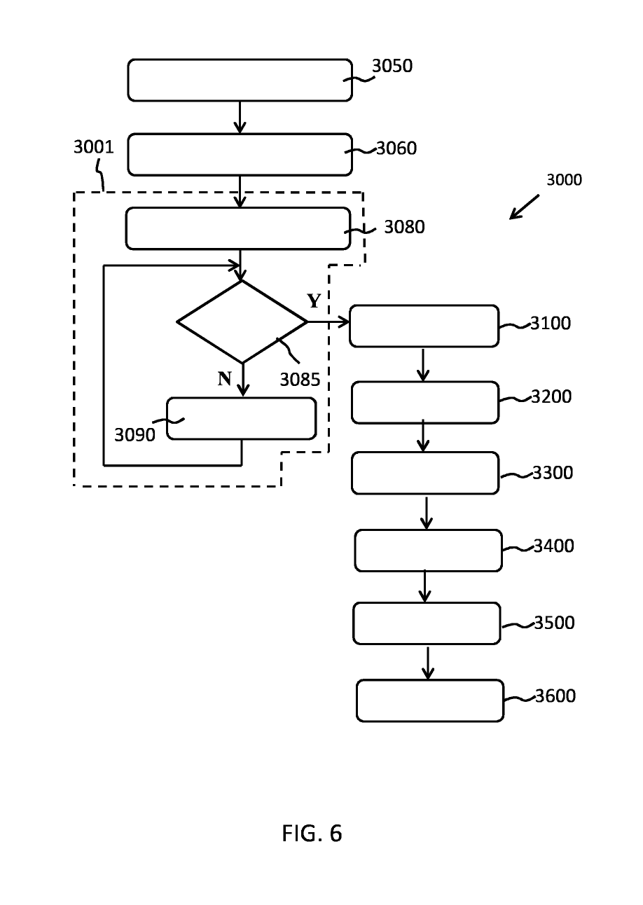

Turning now to FIG. 6, there is depicted a flowchart of an example embodiment of a method 3000 for indicating a condition of a patient.

In a preferred embodiment, the method 3000 comprises a first branch 3001 in which portable computing devices in the vicinity of the layperson establish one or more connections with each other, and a second branch in which the medical guidance system is used to generate an indicator of a condition of a patient in response to processed vital signs information and patient-specific reference information for the vital signs information. It is noted for the avoidance of doubt that the first branch 3001 may be omitted from the method 3000 in case of the portable computing device 100 in the vicinity of the layperson communicating to a remote server or processing resource, for example.

The method 3000 may start in step 3050 by the discovery of a suspected victim of an event by a layperson carrying a portable computing device according to an embodiment. The layperson may activate a condition guidance mode of the portable computing device 100, e.g. by providing the device 100 with a command that can be recognized by the device as an instruction to activate the condition guidance mode. This may prompt the portable computing device to generate a first responder call to emergency services, in order to direct the emergency services to the location of the patient. To this end, the first responder call may be an automated call comprising global positioning information, which information may be obtained from a global positioning unit within the portable computing device 100 or a global positioning unit in communication with the portable computing device 100, e.g. a smart device such as a smart phone or a smart watch comprising a global positioning unit, which smart device is wirelessly linked to the portable computing device 100. Alternatively, the first responder call is generated by the rescuer, e.g. by issuing an instruction to the portable computing device 100 to generate the call.

Next, the method 3000 progresses to step 3060 in which the portable computing device 100 searches in the vicinity of the patient 1000 for devices including a sensor 200 for detecting vital signs (such as breathing characteristics) of the victim or a sensor for sensing the environment within which a victim is located. Such devices may for instance be detected by identifying a wireless signal generated by these devices or by identifying a global positioning tracking signal, e.g. a GPS tracker signal, a GRS signal, a GLONASS signal or the like. These sensors may be incorporated in portable computing devices (e.g. smart devices such as a smart watch or a smart phone) carried or worn by the patient 1000, the rescuer or bystanders. The available devices may be recognized in any suitable manner, for instance by recognizing the device type and make (product number), e.g. from an interrogation of the devices in the vicinity of the patient 1000, and comparing the identified device with a stored database of known devices with the desired sensor functionality, or by requesting the smart devices in the vicinity of the patient 1000 to indicate which sensors are available for linking and then linking to the relevant sensors.

Next, in step 3080, the portable computing device 1000 attempts to establish a wireless connection using any suitable wireless communication protocol as previously explained to the identified devices to be incorporated in the medical guidance system. In step 3085 it is checked if the wireless connections between the identified devices and the portable computing device 100 have been established. If for some reason some of the wireless connections have not been established, the method 3000 proceeds to step 3090 in which the layperson is alerted that some of the wireless connections could not be established, such that the layperson can attempt to adjust the affected devices, for instance by enabling a wireless communication mode of these devices. This may involve asking a bystander to activate the wireless communication mode in case the device has been provided by the bystander. The alert may be displayed on a display module of the portable computing device 100 for example. The method 3000 then returns to step 3085 to check if all devices have established a wireless communication link with the portable computing device 100.

Once all devices are wirelessly connected to the portable computing device 100, the method 3000 proceeds to step 3100 in which vital signs information from at least one sensor is received by the data processor of the portable computing device. For example, data provided by the one or more devices comprising a sensor for monitoring vital signs of the patient may be communicated to the portable computing device. In an embodiment, the data processor may apply weighting factors, e.g. signal quality indicators, to the vital signs data received from the one or more sensors 200, in order to improve the reliability of subsequent processing.

Next, in step 3200, environmental and/or contextual information for the patient from at least one sensor for monitoring environmental or contextual conditions is provided to the portable computing device 100. For example, data from a camera and microphone of a smartphone may be processed in accordance with image and sound classification algorithms incorporating face, object and sound recognition. This processing may be assisted by processing undertaken via a distributed processing environment (such as the ‘cloud’ for example). Also, additional environmental information may be provided from other services, e.g. local weather conditions, so that it can be taken into account by subsequent processing. Thus, in step 3200, various types of environmental information or contextual information, including: location information; weather information; and hazard information may be received by the data processor of the portable computing device.

Then, in step 3300, the environmental and/or contextual information for the patient is communicated from the portable computing device 100 to a database 300 that stores a plurality of patient-specific reference values, each reference value corresponding to a different environmental and/or contextual condition.

In response to the environmental and/or contextual information for the patient received from said portable computing device 100, in step 3400, a patient-specific reference value among the plurality of patient-specific reference values is selected.

In this example, identification information of the patient, such as an identification number or name for example, is also communicated to the remote database. Using such identification information, reference values for the vital signs information which are specific to the identified patient and to a specific environmental and/or contextual condition may be retrieved from the database and communicated to the portable computing device 100. In this way, the selected reference value for the vital signs information received can be made not only environment and/or context-specific, but also patient-specific.

Next, in step 3500, the selected patient-specific reference value for the vital signs information of the patient is provided to the portable computing device 100 from the database 300.

The method then proceeds to step 3600 in which the data processor of the portable computing device 100 processes the vital signs information, the selected patient-specific reference value, and the environmental and/or contextual information so as to generate an indicator of a condition of the patient. Thus, an indicator of patient condition may be generated using vital signs information and the selected patient-specific reference value that can be used to calibrate or adjust the vital signs information so as to cater for patient-specific characteristics (such as age, weight, height, fitness level, etc.). The generated indicator may be further based on environmental and/or contextual information that is representative of environmental and/or contextual conditions (such as weather, location, etc.) that may have an impact on a vital sign of the patient. In this way, an indicator of patient condition that is more accurate and/or patient-specific can be obtained, and this may be achieved from monitoring or sensing vital signs in a simple or non-intrusive manner. The need for complex medical equipment, medical training, and/or intrusive vital sign detection/sensing may therefore be avoided by embodiments.

In some embodiments, the method 3000 may then loop back to step 3100 to perform repeated monitoring of vital signs of the patient 1000. In this manner, the method 3000 may continue until emergency services arrive on the scene, upon which the method 3000 may terminate.

At this point, it is noted that the above described embodiment of the method 3000 is merely an example embodiment of this method and that several extensions thereto and/or variations thereon will be immediately apparent to the skilled person.

For example, the method 3000 may be extended by the detection of a portable defibrillation device in the vicinity of the patient 1000, e.g. in the possession of one of the bystanders, which detection for instance may take place in step 3060 of the method 3000 by way of non-limiting example, in which case the method may be extended by including instructions for automatically operating the defibrillation device based on an generated indicator of a patient's condition. For instance, instructions for operating and/or controlling the defibrillation device may be directly transferred from the portable computing device to the defibrillation device for automatic operation to be implemented.

Other suitable extensions and variations to the above disclosed embodiments will be apparent to the skilled person.

Aspects of the present invention may be embodied as a medical guidance method or system at least partially embodied by a portable computing device or distributed over separate entities including a portable computing device. Aspects of the present invention may take the form of a computer program product embodied in one or more computer-readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. Such a system, apparatus or device may be accessible over any suitable network connection;

for instance, the system, apparatus or device may be accessible over a network for retrieval of the computer readable program code over the network. Such a network may for instance be the Internet, a mobile communications network or the like. More specific examples (a non-exhaustive list) of the computer readable storage medium may include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of the present application, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out the methods of the present invention by execution on the processor of the portable computing device may be written in any combination of one or more programming languages, including an object oriented programming language such as Java, Smalltalk, C++ or the like and conventional procedural programming languages, such as the “C” programming language or similar programming languages. The program code may execute entirely on the processor as a stand-alone software package, e.g. an app, or may be executed partly on the processor and partly on a remote server. In the latter scenario, the remote server may be connected to the portable computing device 100 through any type of network, including a local area network (LAN) or a wide area network (WAN), or the connection may be made to an external computer, e.g. through the Internet using an Internet Service Provider.

Aspects of the present invention are described above with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions to be executed in whole or in part on the data processor of the portable computing device, such that the instructions create means for implementing the functions/acts specified in the flowchart and/or block diagram block or blocks. These computer program instructions may also be stored in a computer-readable medium that can direct the cardiopulmonary resuscitation guidance system including the portable computing device to function in a particular manner.

The computer program instructions may be loaded onto a data processor to cause a series of operational steps to be performed on the portable computing device 100, to produce a computer-implemented process such that the instructions which execute on the data processor to provide processes for implementing the functions/acts specified in the flowchart and/or block diagram block or blocks. The computer program product may form part of a cardiopulmonary resuscitation guidance system including a portable computing device, e.g. may be installed on the cardiopulmonary resuscitation guidance system.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. The word “comprising” does not exclude the presence of elements or steps other than those listed in a claim. The word “a” or “an” preceding an element does not exclude the presence of a plurality of such elements. The invention can be implemented by means of hardware comprising several distinct elements. In the device claim enumerating several means, several of these means can be embodied by one and the same item of hardware. The mere fact that certain measures are recited in mutually different dependent claims does not indicate that a combination of these measures cannot be used to advantage.

Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

1. A method of generating with a processor an indicator of a condition of a patient, the method comprising:
receiving, on a processor of a portable computing device, vital signs information from at least one sensor for monitoring vital signs of the patient;
receiving, on said processor, environmental and/or contextual information for the patient from at least one environmental sensor for monitoring environmental and/or contextual information;
communicating the environmental and/or contextual information for the patient from said processor to a database that stores a plurality of patient-specific reference values, each reference value corresponding to a different environmental and/or contextual condition;
selecting, on said database, a patient-specific reference value among the plurality of patient-specific reference values in response to the environmental and/or contextual information for the patient received from said processor;
receiving, on said processor, the selected patient-specific reference value for the vital signs information of the patient from said database; and
processing, on said processor, the vital signs information and the selected patient-specific reference value so as to generate an indicator of a condition of the patient.

2. The method of claim 1, wherein processing the vital signs information and the selected patient-specific reference value further comprises processing the environmental and/or contextual information for the patient.

3. The method of claim 1, wherein the environmental and/or contextual information comprises at least one of the following: location information; weather information; and hazard information.

4. The method of claim 1, wherein selecting the patient-specific reference value comprises:
determining an environmental and/or contextual condition based on the received environmental and/or contextual information; and
selecting a stored patient-specific reference value corresponding to an environmental and/or contextual condition that is closest to the determined environmental and/or contextual condition.

5. The method of claim 1, wherein the database is provided by a portable computing device carried by the patient.

6. The method of claim 1, further comprising:
receiving, on said processor, identification information of the patient for identifying the patient; and
passing the identification information to the database, so as to enable the selected reference value for the vital signs information received to be patient-specific.

7. The method of claim 1, wherein said vital signs information comprises at least one of: breathing information; blood pressure information; heart rate information; blood glucose information; oxygen saturation information; body temperature information; and pulse information.

8. A computer program product comprising computer program code means adapted to perform all of the steps of the method claim 1 when said program is run on a portable computing device having a processor.

9. A medical guidance system for generating an indicator of a condition of a patient, the system comprising a portable computing device and a database accessible to the portable computing device;
wherein the database stores a plurality of patient-specific reference values for vital signs information of the patient, each reference value corresponding to a different environmental and/or contextual condition;
wherein the portable computing device comprises a processor adapted to:
receive vital signs information from at least one sensor for monitoring vital signs of the patient;
receive environmental and/or contextual information for the patient from at least one environmental sensor for monitoring environmental and/or contextual information;
transmit the environmental and/or contextual information for the patient to the database;
receive selected patient-specific reference value for the vital signs information of the patient from the database; and
process the vital signs information and the selected patient-specific reference value so as to generate an indicator of a condition of the patient; and
wherein the database is adapted to:
receive the environmental and/or contextual information for the patient from the processor;
select a patient-specific reference value among the plurality of patient-specific reference values in response to the received environmental and/or contextual information for the patient; and
transmit the selected patient-specific reference value for the vital signs information of the patient to the processor.

10. The medical guidance system of claim 9 wherein the processor is further adapted to process the vital signs information, the selected patient-specific reference value and the environmental and/or contextual information so as to generate an indicator of a condition of the patient.

11. The medical guidance system of claim 9, wherein the database is further adapted to:
determine an environmental and/or contextual condition based on the received environmental and/or contextual information; and
select a stored patient-specific reference value corresponding to an environmental and/or contextual condition that is closest to the determined environmental and/or contextual condition.

12. The medical guidance system of claim 9, wherein the portable computing device further comprises a patient identification unit adapted to obtain identification information of the patient for identifying the patient, and wherein the processor is adapted to pass the identifying information to the database, such that the selected reference value for the vital signs information received is patient-specific.

13. The medical guidance system of claim 9, wherein the portable computing device further comprises at least one sensor adapted to monitor a vital sign of the patient.

14. The medical guidance system of claim 9, wherein the portable computing device further comprises an automatic defibrillator adapted to automatically defibrillate a patient based on the generated indicator of the condition of the patient.

15. The medical guidance system of claim 9, further
comprising at least one environmental sensor for monitoring environmental and/or contextual information for the patient.
